(19) United States
(12) Patent Application Publication
(10) Pub. No.: US 2026/0224465 A1
(43) Pub. Date: Aug. 6, 2026

(54) COMPOSITIONS COMPRISING A RETINOID AND A PROCESSED OAT INGREDIENT

(71) Applicant: Kenvue Brands LLC, Summit, NJ (US)

(72) Inventors: Ramine Parsa, Lawrenceville, NJ (US); Wen-Hwa Ting Li, Skillman, NJ (US); Daphne Meza, Lansdale, PA (US); Ruchi Patel, Skillman, NJ (US)

(21) Appl. No.: 19/147,490

(22) PCT Filed: Jan. 11, 2024
(86) PCT No.: PCT/IB2024/050294

§ 371 Date: Jul. 11, 2025

Related U.S. Application Data
(60) Provisional application No. 63/479,429, filed on Jan. 11, 2023.

(51) Int. Cl. A61K 8/67; A61K 8/9794; A61Q 19/02; A61Q 19/08

(52) U.S. Cl. CPC A61K 8/671; A61K 8/9794; A61Q 19/02; A61Q 19/08

(57) ABSTRACT

The invention discloses a composition with an anti-primary-aging effect and an application of the composition in cosmetics, and relates to the technical field of cosmetics. The composition comprises encapsulated retinol, retinol palmitate, carnosine, ascorbyl glucoside, nicotinamide, a citrus peel extract, hydrolyzed sodium hyaluraonate, sodium hyaluronate, a sodium hyaluronate cross-linked polymer, beta-oat glucan and a European horse-chestnut seed extract. The composition with the anti-primary-aging effect starts from multiple targets of wrinkle resistance, saccharification resistance, oxidation resistance, melanin fading and brightening, moisturizing, skin refining, defense enhancement, circulation promoting and toxin expelling and the like, and primary-aging symptoms are prevented and improved in a multi-dimensional mode. The cosmetic provided by the invention contains the composition with the anti-primary-aging effect, has the effects of improving and enhancing the comprehensive state of the skin, and is beneficial to restoring the health state of the skin, resisting the primary aging of the skin and delaying the aging.

### FIELD

The present invention generally relates to compositions suitable for use on skin and particularly compositions comprising retinol and a processed oat ingredient.

### BACKGROUND

The human skin is subject to certain aging processes, some of which are attributable to intrinsic processes (e.g. chronoaging) and some of which are attributable to exogenous factors (e.g. photo-aging). In addition, temporary or even lasting changes to the skin can occur, such as acne, greasy or dry skin, keratoses, rosacea, light-sensitive, inflammatory, erythematous, and allergic or autoimmune-reactive reactions, such as dermatosis and photodermatosis.

The consequences of the above-mentioned ageing processes can include thinning of the skin, weaker interlacing of epidermis and dermis, and a reduction in the number of cells and the supplying blood vessels. These consequences are often undesirable, and individuals suffering from these issues will look to topical treatments to address them.

Retinoids have been used for treating skin conditions caused by intrinsic aging, exogenous factors, acne or skin diseases. However, retinoids can be difficult to formulate with because many are susceptible to photodegradation and retinoids can be harsh on the skin of some individuals. Thus, there is an ongoing need and interest in ways of enhancing or finding alternative products/ingredients which activate the same or similar biological pathways as retinoids.

### SUMMARY

Accordingly, one aspect of the invention pertains to skincare composition comprising a retinoid; and a processed oat ingredient. In one or more embodiments, the ratio of retinoid to processed oat ingredient is about 0.000001:10 about 10:1. In some embodiments, the retinoid is present in an amount ranging from about 0.000001% to about 10% by total weight of the composition. In one or more embodiments, the retinoid is selected from the group consisting of retinol, retinal and retinol esters. In some embodiments, the processed oat ingredient is selected from the group consisting of fermented oat, colloidal oat, oat extract, oat oil, and combinations thereof. In one or more embodiments, the processed oat ingredient is selected from the group consisting of fermented oat, colloidal oat, and combination thereof. In some embodiments, the processed oat ingredient is present in an amount ranging from about 0.001 to about 10% by total weight of the composition. In one or more embodiments, the composition further comprises an ingredient selected from the group consisting of surfactants, chelating agents, emollients, humectants, conditioners, preservatives, opacifiers, fragrances, and combinations of two or more thereof. In some embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

Another aspect of the invention pertains to for treating skin comprising topically applying to skin any of the compositions described herein. In one or more embodiments, the method is a method for treating signs of aging, treating acne, smoothing skin texture, or brightening the skin.

Another aspect of the invention pertains to a skincare composition comprising:
a. about 0.01 wt. % to about 1.5 wt. % retinol by total weight of the composition; and
b. about 0.1 wt. % to about 2.5 wt. % by total weight of the composition of a processed oat ingredient selected from the group consisting of fermented oat, colloidal oat, and combinations thereof.

Yet another aspect of the invention pertains to a method for increasing CRABP2 expression in skin cells, the method comprising topically applying to skin a skincare composition comprising a processed oat ingredient. In one or more embodiments, the method is a method for treating signs of aging, treating acne, smoothing skin texture, or brightening the skin. In some embodiments, the processed oat ingredient is selected from the group consisting of fermented oat, colloidal oat, oat extract, oat oil, and combinations thereof. In one or more embodiments, the processed oat ingredient is present in an amount ranging from about 0.001% to about 10% by total weight of the composition. In some embodiments, the skincare composition is substantially free of retinol. In one or more embodiments, the skincare composition is substantially free of a retinoid. In some embodiments, the skincare composition is free of a retinoid.

### DETAILED DESCRIPTION

It is believed that one skilled in the art can, based on the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” includes the endpoints 4 and 9.

As used herein, a composition that is “essentially free” or “substantially free” of an ingredient means the composition that has about 2% or less of that ingredient by weight based on the total weight of the composition. Preferably, a composition that is essentially free of an ingredient has about 1% or less, more preferably about 0.5% or less, more preferably about 0.1% or less, more preferably about 0.05 or less, more preferably about 0.01% or less by weight based on the total weight of composition of the ingredient. In certain more preferred embodiments, a composition that is essentially free of an ingredient is free of the ingredient, i.e. has none of that ingredient in the composition.

As used herein, “cosmetically/dermatologically acceptable” means that the ingredients which the term describes are suitable for use in contact with tissues (e.g., the skin or hair) without undue toxicity, incompatibility, instability, irritation, allergic response, and the like. As will be recognized by one of skill in the art, cosmetically/dermatologically acceptable salts are acidic/anionic or basic/cationic salts.

As used herein, the term “safe and effective amount” means an amount of the compound, extract or of the composition sufficient to induce the desired effect, but low enough to avoid serious side effects. The safe and effective amount of the compound, extract, or composition will vary with e.g. the age, health and environmental exposure of the end user, the duration and nature of the treatment, the specific extract, ingredient, or composition employed, the particular pharmaceutically-acceptable carrier utilized, and like factors.

As used herein, the term “about” refers to within 5% weight, within 4% weight, within 3% weight, within 2.5% weight, within 2% weight, or within 1% weight of a disclosed value.

In general, IUPAC nomenclature rules are used herein and according to the following term definitions.

The term “substituted,” refers to a core molecule in which one or more hydrogen atoms have been replaced with that amount of substituents allowed by available valences. Substitution is not limited to the core molecule, but may also occur on a substituent radical, whereby the radical becomes a linking group.

Acceptable salts from inorganic bases include, for example, sodium or potassium salts, and the like. Acceptable salts from organic bases include, for example, salts formed with primary, secondary, or tertiary amines, and the like.

One or more aspects of the invention pertain to compositions and methods of activating retinoic pathways on the skin. It has been surprisingly discovered that processed oat ingredients are able to accomplish this (e.g., through increase of CRABP2 expression). It has further been surprisingly discovered that processed oat ingredients combined with retinol can have a synergistic effect in terms of activating retinoic pathways.

Accordingly one aspect of the invention pertains to a skincare composition comprising (a) retinol; and (b) a processed oat ingredient. Such compositions have been shown to exhibit synergy in a variety of respects compared to the ingredients alone.

Another aspect of the invention pertains to a method for increasing CRABP2 expression in skin cells, the method comprising topically applying to skin a skincare composition comprising a processed oat ingredient. Because the processed oat ingredient (such as colloidal oat or fermented oat) is capable of increasing CRABP2 on its own, in some embodiments, the skincare composition is substantially free of retinol. In further embodiments, the skincare composition is substantially free, or free, of a retinoid.

### Retinoid

The retinoid as used herein, refers to a class of compounds that possess the biological activity of Vitamin A in the skin. Examples of retinoids include, but are not limited to, retinol, retinaldehyde, retinoic acid, retinyl palmitate, isotretinoin, tazarotene, bexarotene and adapalene. In certain preferred embodiments, the retinoid is retinol. More preferred are retinol, retinal, or mixtures thereof. Most preferred is retinol. These compounds are well known in the art and are commercially available from a number of sources, e.g., Sigma Chemical Company (St. Louis, Mo.), and Boerhinger Mannheim (Indianapolis, Ind.).

In one embodiment, the retinoid is non-encapsulated. That means the retinoid is not contained in or absorbed onto another material. Advantageously, the retinol is not in the form of the RetiSTAR® commercialized by BASF, since it does not need to be stabilized before being used in the composition according to the present invention.

When both are present, the ratio of retinoid to processed oat ingredient may range from about 0.000001:10 to about 10:1. In further embodiments, the ratio of retinoid to processed oat ingredient may range from about 0.000001:10 to about 2:1. In yet further embodiments, the ratio of retinoid to processed oat ingredient is about 0.000006:1 or about 1:10.

The retinoid (e.g., retinol) may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, or 10 wt %. In one or more embodiments, the retinoid (e.g., retinol) may be present in amounts ranging from about 0.001 to about 5 wt %. In further embodiments, the retinoid (e.g., retinol) may be present in amounts ranging from about 0.01 to about 3 wt %. In one or more embodiments, the retinoid (e.g., retinol) may be present in amounts ranging from about 0.01 to about 2 wt %. In further embodiments, the retinoid (e.g., retinol) may be present in amounts ranging from about 0.05 to about 1.5 wt %. In yet further embodiments, the retinoid (e.g., retinol) may be present in amounts ranging from about 0.05 to about 0.5 wt %.

### Processed Oat Ingredient

As used herein, the term “processed oat ingredient” refers to an ingredient that is typically derived from a part of the oat plant (Avena sativa). Said ingredient can be either a processing (e.g., an extract, milling, fermenting) of one or more parts of the oat plant (e.g., grain, leaf, stem, seed) (e.g., an extract) or can be a molecule found in the oat plant (e.g., beta-glucan, flavonoids, avenanthramides, lipids, peptides, etc.). The definition is intended to cover processed oat ingredients which are derived from sources other than oat (e.g., from another plant or chemically synthesized), but are otherwise associated with oat. In one or more embodiments, the processed oat ingredient is selected from the group consisting of oat extract, colloidal oatmeal (used interchangeably with oat flour), oat bran, oat protein, oat peptide, oat oil, fermented oat, avenanthramides, beta-glucan, modified oat grain material (e.g., chemically, enzymatically-, microorganism-modified), and combinations thereof. As used herein, “colloidal oatmeal” means the powder resulting from the grinding and further processing of whole oat grain meeting United States Standards for Number 1 or Number 2 oats. The colloidal oatmeal has a particle size distribution as follows: not more than 3 percent of the total particles exceed 150 micrometers in size and not more than 20 percent of the total particles exceed 75 micrometers in size. Examples of suitable colloidal oatmeals include, but are not limited to, “Tech-0” available from the Beacon Corporation and colloidal oatmeals available from Quaker. In one or more embodiments, the processed oat ingredient comprises oat extract, colloidal oatmeal, and oat oil. In some embodiments, the processed oat ingredient comprises oat extract. In one or more embodiments, the processed oat ingredient comprises colloidal oatmeal. In some embodiments, the processed oat ingredient comprises oat oil. In some embodiments, the processed oat ingredient is selected from the group consisting of oat extract, colloidal oatmeal, oat oil and combinations thereof. In some embodiments, the processed oat ingredient comprises oat extract, colloidal oatmeal, and oat oil. In one or more embodiments, the processed oat ingredient comprises avenanthramides. In some embodiments, the processed oat ingredient comprises fermented oat. In one or more embodiments, the processed oat ingredient comprises beta-glucan.

In further embodiments, the processed oat ingredient is selected from the group consisting of fermented oat, colloidal oat, oat extract, oat oil, and combinations thereof. In yet further embodiments, the processed oat ingredient is selected from the group consisting of fermented oat, colloidal oat, oat extract, and combinations thereof.

The processed oat ingredient may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the processed oat ingredient may be present in amounts ranging from about 0.001 to about 10 wt %. In further embodiments, the processed oat ingredient may be present in amounts ranging from about 0.01 to about 8 wt %. In one or more embodiments, the processed oat ingredient may be present in amounts ranging from about 0.1 to about 6 wt %. In further embodiments, the processed oat ingredient may be present in amounts ranging from about 0.2 to about 5 wt %.

In embodiments where the processed oat ingredient comprises colloidal oat, the colloidal oat may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the colloidal oat may be present in amounts ranging from about 0.001 to about 10 wt %. In further embodiments, the colloidal oat may be present in amounts ranging from about 0.01 to about 8 wt %. In one or more embodiments, the colloidal oat may be present in amounts ranging from about 0.1 to about 5 wt %. In further embodiments, the colloidal oat may be present in amounts ranging from about 0.5 to about 4 wt %. In one or more embodiments, the colloidal oat may be present in amounts ranging from about 0.5 to about 2 wt %, or about 1 wt %. In further embodiments, the colloidal oat may be present in amounts ranging from about 2 to about 4 wt %, or about 3 wt %.

In embodiments where the processed oat ingredient comprises fermented oat, the fermented oat may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the fermented oat may be present in amounts ranging from about 0.001 to about 10 wt %. In further embodiments, the fermented oat may be present in amounts ranging from about 0.01 to about 8 wt %. In one or more embodiments, the fermented oat may be present in amounts ranging from about 0.1 to about 6 wt %. In further embodiments, the fermented oat may be present in amounts ranging from about 0.5 to about 5 wt %.

In embodiments where the processed oat ingredient comprises oat extract, the oat extract may be present in amounts ranging from about 0.00001, 0.0001, 0.001, 0.01, 0.1, 0.2, 0.3, 0.4, 0.5, 0.75, 1, 1.5 or 2 to about 0.05, 0.5, 1, 1.5, 2, 2.5, 3, 4, 5, 6, 7, 8, 9, 10, 11, 12, 13, 14 or 15 wt %. In one or more embodiments, the oat extract may be present in amounts ranging from about 0.001 to about 10 wt %. In further embodiments, the oat extract may be present in amounts ranging from about 0.01 to about 8 wt %. In one or more embodiments, the oat extract may be present in amounts ranging from about 0.1 to about 5 wt %. In further embodiments, the oat extract may be present in amounts ranging from about 0.5 to about 4 wt %.

### Methods

Another aspect of the invention pertains to a method for treating skin, the method comprising topically applying to skin a composition in accordance with one more embodiments of the invention. As used herein, “treatment” or “treating” means the amelioration, prophylaxis, or reversal of a condition, disease, or disorder, or at least one discernible symptom thereof. In one embodiment, “treatment” or “treating” refers to an amelioration, prophylaxis, or reversal of at least one measurable physical parameter related to the condition, disease, or disorder being treated, not necessarily discernible in or by the subject being treated. In another embodiment, “treatment” or “treating” refers to inhibiting or slowing the progression of a condition, disease, or disorder, either physically, e.g., stabilization of a discernible symptom, physiologically, e.g., stabilization of a physical parameter, or both. In another embodiment, “treatment” or “treating” refers to delaying the onset of a condition, disease, or disorder.

The compositions/compounds described herein may be used in treating skin for treating acne, treating the signs of aging (e.g., wrinkles), improving skin barrier function and/or lightening skin. In one or more embodiments, said treatment is for a subject who has a condition or a history of a condition selected from the group consisting of atopic dermatitis, rosacea, seborrheic dermatitis, psoriasis, dry skin, flaky skin.

Compositions of the invention are suitable for improving the texture of skin or improving the firmness of skin, or any of the conditions/symptoms described below.

As used herein, “improving the texture of skin” means the smoothing of the surface of the skin to remove either bumps or crevasses on the skin surface.

As used herein, “improving the firmness of skin” means the enhancing of the firmness or elasticity of the skin, preventing the loss of firmness or elasticity of skin, or preventing or treating sagging, lax and loose skin.

As used herein, “loss of elasticity” includes loss of elasticity or structural integrity of the skin or tissue, including but not limited to sagging, lax and loose tissue. The loss of elasticity or tissue structure integrity may be a result of a number of factors, including but not limited to disease, aging, hormonal changes, mechanical trauma, environmental damage, or the result of an application of products, such as a cosmetics or pharmaceuticals, to the tissue.

As used herein, “uneven skin” means a condition of the skin associated with diffuse or mottled pigmentation, which may be classified as hyperpigmentation, such as post-inflammatory hyperpigmentation.

As used herein, “blotchiness” means a condition of the skin associated with redness or erythema.

As used herein, “age spots” means a condition of the skin associated with discrete pigmentation, e.g., small areas of darker pigmentation that may develop on the face as well as the hands.

Signs of skin aging also include the presence of diminished skin thickness, and abnormal or diminished synthesis of collagen, glycosaminoglycans, proteoglycans, elastin, or glycoproteins including fibronectin. In one embodiment, the sign of aging is selected from the abnormal or diminished synthesis of collagen, glycosaminoglycans, proteoglycans, elastin, or glycoproteins including fibronectin. In another embodiment, the sign of skin aging is diminished synthesis of collagen or elastin.

Examples of skin aging that may be treated by topical use of the compositions of this invention include, but are not limited to, wrinkles on the skin. As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

As used herein, “topical use” and “topically applying” means directly laying on or spreading on the skin, hair, or nail, e.g., by use of the hands or an applicator such as a wipe.

The compositions are also suitable for treating or preventing acne. As used herein, “acne” refers to disorders resulting from the actions of hormones and other substances on the sebaceous glands and hair follicles, typically leading to clogged pores and the formation of inflammatory or non-inflammatory lesions on the skin. Specifically, it relates to blemishes, lesions, or pimples, pre-emergent pimples, blackheads, and/or whiteheads. As used herein, a “pre-emergent pimple” is an inflamed follicle that is not visually apparent on the surface of the skin with the naked eye (e.g., as a lesion).

The compositions of the invention are also suitable for treating or preventing rosacea. As used herein, “rosacea” means skin with persistent erythema with or without papules, pustules, or nodules.

The compositions of the invention are also suitable for reducing epidermal hyperkeratinzation. Accordingly, the composition may be used for treatment or prevention of conditions characterized by hyperkeratinzation, such as acne or warts.

In one or more embodiments, one or more of the methods described herein modify the expression of one or more biomarkers. For example, in one or more embodiments, the method may be a method of increasing CRABP2, HAS2, or HBEGF expression in skin. In further embodiments, the method is a method for increasing CRABP2 expression in skin cells, the method comprising topically applying to skin a skincare composition comprising a processed oat ingredient.

CRABP2 is well-established in literature as a highly sensitive marker of retinoid bioactivity and potency (Elder J T, Cromie M A, Griffiths CEM, Chambon P, Voorhees J J. Stimulus-selective induction of CRABP-II mRNA: A marker for retinoic acid action in human skin. J Invest Dermatol. 1993;100 (4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18;11 (6): 1619-1632). HAS2 is the hyaluronic acid synthase gene that produces hyaluronic acid, which is associated with skin hydration and plumping and is indirectly induced by retinol. Skin plumpness confers antiaging or youthful appearance. Heparin-binding epidermal growth factor (HbEGF). HbEGF is a marker of cell proliferation, which is a feature associated with retinol.

The embodiments described herein may be combined in any suitable combination. For example, an exemplary embodiment pertains to a skincare composition comprising:
a. about 0.01 wt. % to about 1.5 wt. % retinol by total weight of the composition; and
b. about 0.1 wt. % to about 2.5 wt. % by total weight of the composition of a processed oat ingredient selected from the group consisting of fermented oat, colloidal oat, and combinations thereof.

In further embodiments, such a composition may be in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The compositions described herein may be applied to any skin in need of treatment on the human body. For example, application may be made to any one or more of the skin of the face, neck, chest, back, arms, axilla, hands and/or legs. In certain preferred embodiments, the method comprises applying a composition according to one or more embodiments of the invention to skin of the face.

Any suitable method of applying the extract to the skin in need may be used in accordance with the present invention. For example, the extract may be applied directly from a package to the skin in need, by hand to the skin in need, or may be transferred from a substrate such as a wipe or mask, or a combination of two or more thereof. In other embodiments, the extract may be applied via a dropper, tube, roller, spray, patch or added to a bath or otherwise to water to be applied to the skin, and the like.

In one or more embodiments, the methods of the present invention further comprise the step of leaving the composition in contact with the skin for period of time. For example, in certain preferred embodiments after application, the compound is left in contact with the skin for a period of about 15 minutes or greater. In certain more preferred embodiments, the extract is left in contact with the skin for about 20 minutes or greater, more preferably about 1 hour or greater.

In some embodiments, the method of the present invention comprises a regimen comprising applying the composition to skin multiple times over a selected period of time. For example, in certain embodiments, the present invention provides a method of treating signs of aging comprising applying to skin in need of antiaging a composition in accordance with one or more embodiments of the invention once or twice daily for at least 12 weeks, preferably at least 8 weeks and more preferably for at least 2 weeks.

### Compositions

Any suitable carrier may be used in the compositions of the present invention. Preferably, for a skin care composition, the carrier is a cosmetically-acceptable carrier. As will be recognized by those of skill in the art, cosmetically-acceptable carriers comprise carriers that are suitable for use in contact with the body, in particular the skin for antiaging applications, without undue toxicity, incompatibility, instability, irritation, allergic response, and the like. A safe and effective amount of carrier is from about 50% to about 99.999%, preferably from about 80% to about 99.9%, more preferably from about 99.9% to about 95%, most preferably from about 99.8% to about 98% of the composition. The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cP to about 200,000 cP. Examples of suitable cosmetically-acceptable carriers include cosmetically-acceptable solvents and materials for cosmetic solutions, suspensions, lotions, creams, serums, essences, gels, toners, sticks, sprays, ointments, liquid washes and soap bars, shampoos, hair conditioners, pastes, foams, mousses, powders, shaving creams, wipes, patches, strips, powered patches, microneedle patches, bandages, hydrogels, film-forming products, facial and skin masks, makeup, liquid drops, and the like. These product types may contain several types of cosmetically-acceptable carriers including, but not limited to solutions, suspensions, emulsions such as microemulsions and nanoemulsions, gels, solids, liposomes, other encapsulation technologies and the like. In one or more embodiments, the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

The following are non-limitative examples of such carriers. Other carriers can be formulated by those of ordinary skill in the art. In one embodiment, the carrier contains water. In a further embodiment, the carrier may also contain one or more aqueous or organic solvents. Examples of organic solvents include, but are not limited to: dimethyl isosorbide; isopropylmyristate; surfactants of cationic, anionic and nonionic nature; vegetable oils; mineral oils; waxes; gums; synthetic and natural gelling agents; alkanols; glycols; and polyols. Examples of glycols include, but are not limited to, glycerin, propylene glycol, butylene glycol, pentylene glycol, hexylene glycol, polyethylene glycol, polypropylene glycol, diethylene glycol, triethylene glycol, capryl glycol, glycerol, butanediol and hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms) such as propylene glycol. The organic solvents may be present in the carrier in an amount, based upon the total weight of the carrier, of from about 1 percent to about 99.99 percent (e.g., from about 20 percent to about 50 percent). Water may be present in the carrier (prior to use) in an amount, based upon the total weight of the carrier, of from about 5 percent to about 95 percent (e.g., from about 50 percent to about 90 percent). Solutions may contain any suitable amounts of solvent, including from about 40 to about 99.99%. Certain preferred solutions contain from about 50 to about 99.9%, from about 60 to about 99%, from about 70 to about 99%, from about 80 to about 99%, or from about 90 to 99%.

A lotion can be made from such a solution. Lotions typically contain at least one emollient in addition to a solvent. Lotions may comprise from about 1% to about 20% (e.g., from about 5% to about 10%) of an emollient(s) and from about 50% to about 90% (e.g., from about 60% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7th Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s).

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, or wipe). The composition of the present invention can also be combined with a solid, semi-solid or dissolvable substrate (eg., a wipe, mask, pad, glove or strip).

The compositions of the present invention may further comprise any of a variety of additional cosmetically active agents, although they are preferably formulated to account for use on skin. Examples of suitable additional active agents include: additional skin lightening agents, darkening agents, anti-acne agents, shine control agents, antimicrobial agents such as anti-yeast agents, anti-fungal, and anti-bacterial agents, anti-inflammatory agents, anti-parasite agents, external analgesics, sunscreens, photo-protectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, hydration boosters, efficacy boosters, anti-callous agents, agents for skin conditioning, anti-cellulite agents, fluorides, teeth whitening agents, anti-plaque agents, and plaque-dissolving agents, odor-control agents such as odor masking or pH-changing agents, and the like. Examples of various suitable additional cosmetically acceptable actives include hydroxy acids, benzoyl peroxide, D-panthenol, UV filters such as but not limited to avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, carotenoids, free radical scavengers, spin traps, retinoids and retinoid precursors such as retinol, retinoic acid and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu: Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as oat, aloe vera, Feverfew, Soy, Shiitake mushroom extracts, and derivatives and mixtures thereof.

In one or more embodiments, the compositions of the present invention are skin care compositions that further comprise a at least one skin lightening active agent. Examples of suitable skin lightening active agents include, but are not limited to, tyrosinase inhibitors, melanin-inhibiting agents, melanosome transfer inhibiting agents including PAR-2 antagonists, exfoliants, sunscreens, retinoids, antioxidants, Tranexamic acid, skin bleaching agents, allantoin, opacifiers, talcs and silicas, zinc salts, and the like, and other agents as described in Solano et al. Pigment Cell Res. 2006, 19 (550-571). Examples of suitable tyrosinase inhibitors include but, are not limited to, Vitamin C and its derivatives, Vitamin E and its derivatives, Kojic Acid, Arbutin, resorcinols, hydroquinone, Flavones e.g. Licorice flavanoids, Licorice root extract, Mulberry root extract, Dioscorea Coposita root extract, Saxifraga extract and the like, Ellagic acid, Salicylates and derivatives, Glucosamine and derivatives, Fullerene, Hinokitiol, Dioic acid, Acetyl glucosamine, Magnolignane, combinations of two or more thereof, and the like. Examples of vitamin C derivatives include, but are not limited to, ascorbic acid and salts, Ascorbic Acid-2-Glucoside, sodium ascorbyl phosphate, magnesium ascorbyl phosphate, and natural extract enriched in vitamin C. Examples of vitamin E derivatives include, but are not limited to, alpha-tocopherol, beta, tocopherol, gamma-tocopherol, delta-tocopherol, alpha-tocotrienol, beta-tocotrienol, gamma-tocotrienol, delta-tocotrienol and mixtures thereof, tocopherol acetate, tocopherol phosphate and natural extracts enriched in vitamin E derivatives. Examples of resorcinol derivatives include, but are not limited to, resorcinol, 4-substituted resorcinols like 4-alkylresorcinols such as 4-butyresorcinol (rucinol), 4-hexylresorcinol, phenylethyl resorcinol, 1-(2,4-dihydroxyphenyl)-3-(2,4-dimethoxy-3-methylphenyl)-Propane and the like and natural extracts enriched in resorcinols. Examples of salicylates include, but are not limited to, salicylic acid, acetylsalicylic acid, 4-methoxysalicylic acid and their salts. In certain preferred embodiments, the tyrosinase inhibitors include a 4-substituted resorcinol, a vitamin C derivative, or a vitamin E derivative. In more preferred embodiments, the tyrosinase inhibitor comprises Phenylethyl resorcinol, 4-hexyl resorcinol, or ascorbyl-2-glucoside.

Examples of suitable melanin-degradation agents include, but are not limited to, peroxides and enzymes such as peroxidases and ligninases. In certain preferred embodiments, the melanin-inhibiting agents include a peroxide or a ligninase.

Examples of suitable melanosome transfer inhibiting agents including PAR-2 antagonists such as soy trypsin inhibitor or Bowman-Birk Inhibitor, Vitamin B3 and derivatives such as Niacinamide, Essential soy, Whole Soy, Soy extract. In certain preferred embodiments, the melanosome transfer inhibiting agents includes a soy extract or niacinamide.

Examples of exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion. In certain preferred embodiments, the exfoliant include glycolic acid or salicylic acid.

Examples of sunscreens include, but are not limited to, avobenzone (Parsol 1789), bisdisulizole disodium (Neo Heliopan AP), diethylamino hydroxybenzoyl hexyl benzoate (Uvinul A Plus), ecamsule (Mexoryl SX), methyl anthranilate, 4-aminobenzoic acid (PABA), cinoxate, ethylhexyl triazone (Uvinul T 150), homosalate, 4-methylbenzylidene camphor (Parsol 5000), octyl methoxycinnamate (Octinoxate), octyl salicylate (Octisalate), padimate O (Escalol 507), phenylbenzimidazole sulfonic acid (Ensulizole), polysilicone-15 (Parsol SLX), trolamine salicylate, Bemotrizinol (Tinosorb S), benzophenones 1-12, dioxybenzone, drometrizole trisiloxane (Mexoryl XL), iscotrizinol (Uvasorb HEB), octocrylene, oxybenzone (Eusolex 4360), sulisobenzone, bisoctrizole (Tinosorb M), titanium dioxide, zinc oxide, and the like.

Examples of antioxidants include, but are not limited to, water-soluble antioxidants such as sulfhydryl compounds and their derivatives (e.g., sodium metabisulfite and N-acetyl-cysteine, glutathione), lipoic acid and dihydrolipoic acid, stilbenoids such as resveratrol and derivatives, lactoferrin, and ascorbic acid and ascorbic acid derivatives (e.g., ascobyl-2-glucoside, ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, feverfew, parthenolide-free feverfew, oat extracts, pomelo extract, wheat germ extract, Hesperidin, Grape extract, Portulaca extract, Licochalcone, chalcone, 2,2′-dihydroxy chalcone, Primula extract, propolis, and the like.

The additional cosmetically active agent may be present in a composition in any suitable amount, for example, in an amount of from about 0.0001% to about 20% by weight of the composition, e.g., about 0.001% to about 10% such as about 0.01% to about 5%. In certain preferred embodiments, in an amount of 0.1% to 5% and in other preferred embodiments from 1% to 2%.

A variety of other materials may also be present in the compositions of the present invention. These include, for example, chelating agents, humectants, opacifiers, conditioners, preservatives, fragrances and the like. The compositions may include surfactants, for example, those selected from the group consisting of anionic, non-ionics, amphoteric, cationic, or a combination of two or more thereof.

The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

In one or more embodiments, the present invention comprises applying a compound or composition of the invention via a substrate comprising such material. Any suitable substrate may be used in the present invention. Examples of suitable substrates and substrate materials are disclosed, for example, in U.S. Published Application Nos. 2005/022683 and 2009/0241242 which are incorporated herein by reference in their entirety. In certain preferred embodiments, the substrate is a wipe or a facial mask.

The composition and products containing such compositions of this invention may be prepared using methodology that is well known by an artisan of ordinary skill.

### EXAMPLES

The below examples evaluate various samples including processed oat ingredients, retinol and combinations thereof. Some examples pertain to evaluation of the of retinol-responsive genes such as CRABP2, HbEGF, HAS2, and HAS3. The upregulation of these genes is associated with skin benefits provided by retinoids (e.g., anti-aging effects, reduction of wrinkles, acne, and tone benefits. Some examples pertain to evaluation of the gene for IL-8, an inflammatory mediator interleukin. Expression of the IL-8 gene is associated with increased inflammation.

### Example 1: Bioactivity Assay of CRAPB2, IL8 and HAS2 in Human Dermal Fibroblasts of Fermented Oat and Retinol, Alone and in Combination

CRAPB2, IL8 and HAS2 were evaluated in several samples containing fermented oat, retinol, and combinations of the two in varying amounts. Retinol (BASF, 50C) was prepared by dissolving Retinol in Dimethyl Sulfoxide (DMSO) for a concentration of 20% Retinol. Retinol was then further diluted in DMEM with GlutaMAX media supplemented with 1% FBS and 1X Penn/Strep for a stock concentration of (1.1×10⁻⁴) % and (1.1×10⁻⁵) % Retinol. Since Retinol is 50% active, the percent active in the stock concentrations are (6×10⁻⁵) % and (6×10⁻⁶) %. Fermented Oat (aurafirm P, Oat Cosmetics, Hampshire, United Kingdom; INCI Aqua, Avena sativa (Oat) Kernel Extract, Lactobacillus Ferment, Sodium Benzoate, Potassium Sorbate) was diluted in media to provide 0.2% and 1.0% Fermented Oat solutions. 100 μL of each test solution were added to each well to achieve the final concentrations as listed in Tables 1-3 below.

Primary human dermal fibroblasts were acquired from Lifeline Cell Technology, LLC (Frederick, MD 21701; lot 0967). The donor demographics were as follows: 18 year old, Female, Caucasian, fibroblasts isolated from breast. Fibroblasts were cultured and expanded using Dermal Fibroblast Culture Medium (Zenbio, Inc, Durham, NC 27713). In preparation for the experiment, fibroblasts were seeded at a concentration of 10,000 cells/well in 96-well plates. Cells were treated 6-24 hours upon seeding with DMEM with GlutaMAX media supplemented with 1% FBS and 1X Penn/Strep. Upon 30 hours of treatment, a Cell-to-CT kit (Invitrogen, Waltham, MA) was used to isolate RNA per manufacturer's instructions and converted to cDNA. The cDNA was evaluated by qPCR (using the QuantStudio™ 7 Flex Real-Time PCR System from Applied Biosystems, Waltham, MA). Gene expression assays sold under the tradename TAQMAN (ThermoFisher Scientific, Bridgewater, NJ) for cellular retinoic acid binding protein 2 (CRABP2), interleukin-8 (IL8), hyaluronan synthase 2 (HAS2), and polymerase (RNA) II polypeptide A (POLR2A), were used. The expression of these genes was normalized against the expression of the human POLR2A housekeeping gene. The fold changes were calculated in comparison to the untreated control and two-tailed two-sample Student t-tests (Microsoft Office Excel 2007; Microsoft, Redmond, WA, USA) were performed. The calculation of synergy was performed as follows: Briefly, the resultant fold change over untreated of each ingredient alone was added together, and a 1fold change was subtracted to account for untreated contributions. If the result was smaller than the resultant bioactivity of the ingredient combination, then it was considered synergistic, since the result was greater than the sum of its parts. Results are shown as Average Fold Change over Untreated in Tables 1-3.

TABLE 1
CRABP2 Average Fold Change over Untreated

| | | Average Fold | | |
|---|---|---|---|---|
| Sample | Treatment | Change over | Standard | Synergy |
| No. | CRABP2 | untreated | Deviation | Comparison |
| 1-1 | Untreated | 1.00 | 0.12 | |
| (Comp.) | | | | |
| 1-2 | (3 × 10⁻⁶)% | 1.44 | 0.09 | |
| (Comp.) | Retinol | | | |
| 1-3 | (3 × 10⁻⁵)% | 4.25 | 0.45 | |
| (Comp.) | Retinol | | | |
| 1-4 | 0.1% | 0.91 | 0.21 | |
| (Comp.) | Fermented | | | |
| | Oat | | | |
| 1-5 | 0.5% | 2.47 | 0.88 | |
| (Comp.) | Fermented | | | |
| | Oat | | | |
| 1-6 | 0.1% | 2.62 | 1.26 | (0.91 + 1.44 − |
| | Fermented | | | 1) = 1.35 |
| | Oat + | | | 2.62 > 1.35 |
| | (3 × 10⁻⁶)% | | | |
| | Retinol | | | |
| 1-7 | 0.1% | 3.31 | 0.23 | |
| | Fermented | | | |
| | Oat + | | | |
| | (3 × 10⁻⁵)% | | | |
| | Retinol | | | |
| 1-8 | 0.5% | 4.26 | 0.71 | (2.47 + 1.44 − |
| | Fermented | | | 1) = 2.91 |
| | Oat + | | | 4.26 > 2.91 |
| | (3 × 10⁻⁶)% | | | |
| | Retinol | | | |
| 1-9 | 0.5% | 6.30 | 1.30 | (2.47 + 4.25 − |
| | Fermented | | | 1) = 5.72 |
| | Oat + | | | 6.30 > 5.72 |
| | (3 × 10⁻⁵)% | | | |
| | Retinol | | | |

TABLE 2
IL8 Average Fold Change over Untreated

| | | Average Fold | | |
|---|---|---|---|---|
| Sample | Treatment | Change over | Standard | Synergy |
| No. | IL8 | untreated | Deviation | Comparison |
| 1-1 | Untreated | 1.00 | 0.60 | |
| (Comp.) | | | | |
| 1-2 | (3 × 10⁻⁶)% | 2.05 | 0.70 | |
| (Comp.) | Retinol | | | |
| 1-3 | (3 × 10⁻⁵)% | 18.83 | 6.37 | |
| (Comp.) | Retinol | | | |
| 1-4 | 0.1% | 2.74 | 1.06 | |
| (Comp.) | Fermented | | | |
| | Oat | | | |
| 1-5 | 0.5% | 18.23 | 1.38 | |
| (Comp.) | Fermented | | | |
| | Oat | | | |
| 1-6 | 0.1% | 3.33 | 1.44 | |
| | Fermented | | | |
| | Oat + | | | |
| | (3 × 10⁻⁶)% | | | |
| | Retinol | | | |
| 1-7 | 0.1% | 12.78 | 4.11 | |
| | Fermented | | | |
| | Oat + | | | |
| | (3 × 10⁻⁵)% | | | |
| | Retinol | | | |
| 1-8 | 0.5% | 22.02 | 13.41 | (18.23 + 2.05 − |
| | Fermented | | | 1) = 19.28 |
| | Oat + | | | 22.02 > 19.28 |
| | (3 × 10⁻⁶)% | | | |
| | Retinol | | | |
| 1-9 | 0.5% | 43.58 | 26.56 | (18.23 + 18.83 − |
| | Fermented | | | 1) = 36.06 |
| | Oat + | | | 43.58 > 36.06 |
| | (3 × 10⁻⁵)% | | | |
| | Retinol | | | |

TABLE 3
HAS2 Average Fold Change over Untreated

| | | Average Fold | |
|---|---|---|---|
| Sample | | Change over | Standard |
| No. | Treatment HAS2 | untreated | Deviation |
| 1-1 | Untreated | 1.00 | 0.16 |
| (Comp.) | | | |
| 1-2 | (3 × 10⁻⁶)% | 0.79 | 0.14 |
| (Comp.) | Retinol | | |
| 1-3 | (3 × 10⁻⁵)% | 0.50 | 0.10 |
| (Comp.) | Retinol | | |
| 1-4 | 0.1% | 1.50 | 0.14 |
| (Comp.) | Fermented | | |
| | Oat | | |
| 1-5 | 0.5% | 3.87 | 0.21 |
| (Comp.) | Fermented | | |
| | Oat | | |
| 1-6 | 0.1% | 1.34 | 0.53 |
| | Fermented | | |
| | Oat + | | |
| | (3 × 10⁻⁶)% | | |
| | Retinol | | |
| 1-7 | 0.1% | 0.80 | 0.11 |
| | Fermented | | |
| | Oat + | | |
| | (3 × 10⁻⁵)% | | |
| | Retinol | | |
| 1-8 | 0.5% | 2.75 | 0.29 |
| | Fermented | | |
| | Oat + | | |
| | (3 × 10⁻⁶)% | | |
| | Retinol | | |
| 1-9 | 0.5% | 2.24 | 0.44 |
| | Fermented | | |
| | Oat + | | |
| | (3 × 10⁻⁵)% | | |
| | Retinol | | |

The results indicated fermented oat in combination with retinol resulted in synergistically induced retinoid bioactivity, as measured by CRABP2 in human dermal fibroblast model. CRABP2 is a sensitive biomarker for retinoid bioactivity. CRABP2 is well-established in literature as a highly sensitive marker of retinoid bioactivity and potency (Elder J T, Cromie M A, Griffiths CEM, Chambon P, Voorhees J J. Stimulus-selective induction of CRABP-II mRNA: A marker for retinoic acid action in human skin. J Invest Dermatol. 1993;100 (4), and has been associated with conferring skin anti-aging benefits (Bielli A, Scioli M G, D'Amico F, Tarquini C, Agostinelli S, Costanza G, Doldo E, Campione E, Passeri D, Coniglione F, Orlandi A. Cellular retinoic acid binding protein-II expression and its potential role in skin aging. Aging (Albany NY). 2019 Mar. 18;11 (6): 1619-1632). This data shows the combination of these two ingredients results in a synergistic increase of retinol-like property, and hence antiaging benefits. Additionally, it was surprisingly discovered that the fermented oat alone also provided some CRABP2 expression on its own, which is surprising, as fermented oat is not known to do so.

IL8 is a marker associated with retinoid irritation. With respect to IL8, the results above indicate 0.1% fermented oat induced similar levels of IL8 as (3×10⁻⁶) % retinol and 0.5% fermented oat induced similar levels of IL8 as (3×10⁻⁵) % retinol further confirming the retinoid-like bioactivity of fermented oat. When fermented oat was tested in combination with retinol, the combination synergistically induced IL8, meaning there was a synergistic activation of a pathway associated with retinol.

HAS2 is a marker associated with hyaluronic acid synthase and skin hydration. The results demonstrate that, although Fermented Oat alone was able to induce HAS2, a hyaluronic acid synthase driven hydration synergy was not present when the combinations of Fermented oat and retinol were evaluated under the specific conditions and doses tested. Nevertheless, it is thought that higher doses might be required to observe the synergy for this combination for HAS2 induction, as was seen in the next example.

### Example 2: Bioactivity Assay of CRABP2, HBEGE, IL8, and HAS3 in Human Skin Explants of Samples with Colloidal Oat

Propylene glycol/ethanol (PgETOH) (3:7) (w/w) was used as a vehicle. 1% Colloidal oat (Avena Sativa Kernel Flour, Oat Cosmetics, Southampton, United Kingdom) and 0.1% Retinol solutions were each prepared in PgETOH as shown in Tables 4-7. Combinations were prepared by adding the appropriate amount of test solutions to arrive at the final concentrations as shown.

Abdominal skin samples were obtained from human adults undergoing abdominoplasty surgery. Informed consent was obtained from each patient, and all experimental steps were approved by an institutional review board (IRB). Subcutaneous fat was carefully removed and skin biopsies of 0.93 cm² were prepared under sterile conditions and acclimated in DMEM/F12 (1:1) medium, 2% heat-inactivated fetal bovine serum, 10 μg/mL insulin, 10 ng/mL hydrocortisone, 10 ng/mL EGF, 1x ABAM under a 5% CO₂ humidified atmosphere overnight. Skin explants were treated topically with 4 μL of each formulation for 48 hours.

At the end of the 48 h incubation, the skin biopsies were cut in half and either half of the skin biopsies or the two-halves were each lysed in 600 μL lysis buffer, consisting of 100 parts RLT buffer (RNA purification kit, sold under the tradename RNEASY Mini kit, Qiagen, Valencia, CA), to one part 2-mercaptoethanol inside a reinforced tube with screw cap and o-ring closure, and ceramic beads in the tube for tissue grinding (sold under the tradename PRECELLYS CKMix50-R, Bertin Corp, Rockville, MD). The tubes were shaken 40 seconds at 6300 rpm. RNA was extracted from the solutions using the RNEASY Mini Kit (Qiagen, Valencia, CA) according to manufacturer's instructions and RNA was eluted in 30 μL RNase-free water

Reverse transcription (RT) was performed using the Applied Biosystems High Capacity Reverse Transcription Kit (ThermoFisher Scientific, Bridgewater, NJ). Gene expression assays sold under the tradename TAQMAN for cellular retinoic acid binding protein 2 (CRABP2), heparin-binding epidermal growth factor (HbEGF), hyaluronan synthase 3 (HAS3), interleukin-8 (IL8), polymerase (RNA) II polypeptide A (POLR2A), 18S rRNA (18S), TATA box binding protein (TBP), and master mix sold under the tradename TAQMAN (ThermoFisher Scientific, Bridgewater, NJ). qPCR analysis was performed using the TAQMAN master mix, and run on a real time PCR system sold under the tradename QUANTSTUDIO 7 Flex System (ThermoFisher Scientific, Bridgewater, NJ). The expression of these genes was normalized against either the expression of the human POLR2A, or alternatively against the expression of the human 18S rRNA or TBP “housekeeping” genes. These “housekeeping” genes are widely used controls for normalizing specific gene expression levels because of their invariant expression across tissues, cells, and experimental treatments. The method for normalization involves measuring the expression of an internal reference or “housekeeping” gene, which takes into account the potential error of RNA/cDNA loading, and variation of reverse transcription efficiency. The fold changes were calculated in comparison to the untreated or vehicle controls and two-tailed two-sample Student t-tests (Microsoft Office Excel 2007; Microsoft, Redmond, WA, USA) were performed. Synergies were calculated as described above. Results are shown as Average Fold Change over Untreated in Tables 4-7.

TABLE 4
CRABP2 Average Fold Change over Untreated

| | | Average Fold | | |
|---|---|---|---|---|
| | Treatment | Change over | Standard | Synergy |
| Sample | CRABP2 | untreated | Deviation | Comparison |
| 2-1 | Untreated | 1.02 | 0.24 | |
| (Comp.) | | | | |
| 2-2 | PGEtOH | 0.98 | 0.17 | |
| (Comp.) | | | | |
| 2-3 | 0.1% Retinol | 3.95 | 0.62 | |
| (Comp.) | | | | |
| 2-4 | 1% Colloidal oat | 1.55 | 0.32 | |
| (Comp.) | | | | |
| 2-5 | 0.1% Retinol + | 5.38 | 0.07 | (3.95 + 1.55 − |
| | 1% Colloidal oat | | | 1) = 4.5 |
| | | | | 5.38 > 4.5 |

TABLE 5
HAS3 Average Fold Change over Untreated

| | | Average Fold | | |
|---|---|---|---|---|
| | Treatment | Change over | Standard | Synergy |
| Sample | HAS3 | untreated | Deviation | Comparison |
| 2-1 | Untreated | 1.02 | 0.21 | |
| (Comp.) | | | | |
| 2-2 | PGEtOH | 1.09 | 0.22 | |
| (Comp.) | | | | |
| 2-3 | 0.1% Retinol | 3.05 | 0.48 | |
| (Comp.) | | | | |
| 2-4 | 1% Colloidal oat | 1.39 | 0.32 | |
| (Comp.) | | | | |
| 2-5 | 0.1% Retinol + | 4.11 | 0.14 | (3.05 + 1.39 − |
| | 1% Colloidal Oat | | | 1) = 3.44 |
| | | | | 4.11 > 3.44 |

TABLE 6
HBEGF Average Fold Change over Untreated

| | | Average Fold | | |
|---|---|---|---|---|
| | Treatment | Change over | Standard | Synergy |
| Sample | HBEGF | untreated | Deviation | Comparison |
| 2-1 | Untreated | 1.01 | 0.17 | |
| (Comp.) | | | | |
| 2-2 | PGEtOH | 1.05 | 0.18 | |
| (Comp.) | | | | |
| 2-3 | 0.1% Retinol | 3.50 | 0.82 | |
| (Comp.) | | | | |
| 2-4 | 1% Colloidal oat | 1.68 | 0.56 | |
| (Comp.) | | | | |
| 2-5 | 0.1% Retinol + | 5.05 | 0.34 | (3.50 + 1.68 − |
| | 1% Colloidal Oat | | | 1) = 4.18 |
| | | | | 5.05 > 4.18 |

TABLE 7
IL8 Average Fold Change over Untreated

| | | Average Fold | | |
|---|---|---|---|---|
| | Treatment | Change over | Standard | Synergy |
| Sample | IL8 | untreated | Deviation | Comparison |
| 2-1 | Untreated | 1.04 | 0.36 | |
| (Comp.) | | | | |
| 2-2 | PGEtOH | 2.42 | 0.74 | |
| (Comp.) | | | | |
| 2-3 | 0.1% Retinol | 2.30 | 0.98 | |
| (Comp.) | | | | |
| 2-4 | 1% Colloidal oat | 1.17 | 0.11 | |
| (Comp.) | | | | |
| 2-5 | 0.1% Retinol + | 4.79 | 0.01 | (2.3 + 1.17 − |
| | 1% Colloidal Oat | | | 1) = 2.47 |
| | | | | 4.79 > 2.47 |

As can be seen from the results shown in Tables 4-7, the colloidal oat alone increased retinoid activity, as measured by CRABP2, HAS3, and HBEGF. This is surprising given that oat is not generally known for enhancing retinoid activity. Furthermore, the colloidal oat when in combination with retinol also increased the retinoid activity, and surprisingly did so synergistically. As these are biomarkers associated with anti-aging benefits, it suggests that the colloidal oat alone and especially the combination would be advantageous for providing such anti-aging benefits.

What is claimed is:

1. A skincare composition comprising:
a. a retinoid; and
b. a processed oat ingredient.

2. The skincare composition of claim 1, wherein the ratio of retinoid to processed oat ingredient is about 0.000001:10 about 10:1.

3. The skincare composition of claim 1, wherein the retinoid is present in an amount ranging from about 0.000001% to about 10% by total weight of the composition.

4. The skincare composition of claim 1, wherein the retinoid is selected from the group consisting of retinol, retinal and retinol esters.

5. The skincare composition of claim 1, wherein the processed oat ingredient is selected from the group consisting of fermented oat, colloidal oat, oat extract, oat oil, and combinations thereof.

6. The skincare composition of claim 4, wherein the processed oat ingredient is selected from the group consisting of fermented oat, colloidal oat, and combination thereof.

7. The skincare composition of claim 1, wherein the processed oat ingredient is present in an amount ranging from about 0.001 to about 10% by total weight of the composition.

8. The skincare composition of claim 1, further comprising an ingredient selected from the group consisting of surfactants, chelating agents, emollients, humectants, conditioners, preservatives, opacifiers, fragrances, and combinations of two or more thereof.

9. The skincare composition of claim 1, wherein the composition is in the form of a solution, suspension, emulsion, lotion, cream, serum, gel, stick, spray, ointment, liquid wash, soap bar, shampoo, hair conditioner, paste, foam, powder, mousse, shaving cream, hydrogel, or film-forming product.

10. A method for treating skin comprising topically applying to skin the composition of claim 1.

11. The method of claim 10, wherein the method is a method for treating signs of aging, treating acne, smoothing skin texture, or brightening the skin.

12. A skincare composition comprising:
a. about 0.01 wt. % to about 1.5 wt. % retinol by total weight of the composition; and
b. about 0.1 wt. % to about 2.5 wt. % by total weight of the composition of a processed oat ingredient selected from the group consisting of fermented oat, colloidal oat, and combinations thereof.

13. A method for increasing CRABP2 expression in skin cells, the method comprising topically applying to skin a skincare composition comprising a processed oat ingredient.

14. The method of claim 13, wherein the method is a method for treating signs of aging, treating acne, smoothing skin texture, or brightening the skin.

15. The method of claim 13, wherein the processed oat ingredient is selected from the group consisting of fermented oat, colloidal oat, oat extract, oat oil, and combinations thereof.

16. The method of claim 13, wherein the processed oat ingredient is present in an amount ranging from about 0.001% to about 10% by total weight of the composition.

17. The method of claim 13, wherein the skincare composition is substantially free of retinol.

18. The method of claim 13, wherein the skincare composition is substantially free of a retinoid.

19. The method of claim 13, wherein the skincare composition is free of a retinoid.

19. The method of claim 13, wherein the skincare composition is free of a retinoid.
